(19) United States
(12) Patent Application Publication
(10) Pub. No.: US 2008/0085213 A1
(43) Pub. Date: Apr. 10, 2008

(54) Cartridge-type reagent chip analyzer

(75) Inventors: Tzu-Chiang Wu, Miaoli City (TW); Mao-Ji Lin, Sigang Township (TW); Chien-Ho Chuang, Kaohsiung (TW); Tsung-Kai Chuang, Tainan City (TW); Jiann-Hua Wang, Taipei (TW)

Correspondence Address: BIRCH STEWART KOLASCH & BIRCH, PO BOX 747, FALLS CHURCH, VA, 22040-0747, US

(73) Assignee: Kaiwood Technology Co., Ltd.

(21) Appl. No.: 11/542,179

(22) Filed: Oct. 4, 2006

(51) Int. Cl. G01N 33/48

(52) U.S. Cl. 422/68.1

(57) ABSTRACT

A cartridge-type reagent chip analyzer is provided to analyze various specifications of reagent chips. A primary feature of the present invention is to provide a gear mounted on a base and two gear racks symmetrically meshing with the gear. The gear racks are connected to two guiding components and connected to the base by two springs respectively. When a reagent chip is inserted, the guiding components are pushed apart and guide the reagent chip to enter the analyzer. When the reagent chip is ejected out of the analyzer, the springs force the guiding components back to their original positions.

### BACKGROUND OF THE INVENTION

1. Field of the Invention

The present invention relates to a cartridge-type reagent chip analyzer that can analyze various specifications of reagent chips.

2. The Prior Arts

Drug abuse may lead to physical, social, and psychological harm. For safety reason, certain government agencies and private companies begin to drug test people before a new employee is hired. Sports organizations also test athletes for performance enhancing drugs during the sports events for a fair competition. The majority of drugs can be detected in samples of urine. For some substances, blood samples may be required. Except tests for sophisticated projects that need to use special instruments and spend more time, tests for general projects usually only need to use the reagent, which can get testing results in a short period.

The present testing reagent in common use is a reagent strip having a reagent absorbed on the strip surface, or a reagent chip produced by biotechnology. After the reagent chip is exposed to biological fluid, the reagent develops a color or color intensity corresponding to the presence and/or concentration of the constituents present in the biological fluid being tested. For example, if the biological fluid contains a constituent of an illegal drug, the reagent chip will show color intensity different from the standard color according to the designs of various companies. The color intensity is directly proportional to the concentration of the illegal drug.

In order to read the testing result, a color standard corresponding to different concentration levels for the tested parameters is incorporated into the reagent chip. After the reagent chip is exposed to the biological fluid and appears its color, the color of the reagent chip is then read against the color standard. It is hard to obtain the accurate testing value due to the visual errors of reading the color intensity. Thus, a Charge-Coupled Device (CCD) combined with computer software replaces the visual reading of the reagent chips. The method according to the present invention uses the CCD camera to take the image of the reagent chip and download it into a computer having a color standard comparison program. The color of the reagent chip is converted to a quantitative measurement of the parameter concentration, and compared with the calibrated color on the same strip. Then the testing result may be shown in numerical value, the specific and vivid color or expressed by any other proper ways.

There are a variety of reagent chips, and one of them is a rectangular reagent chip like a floppy disk. Reagent chips soaked with urine are inserted into a slot of the analyzer. An examiner takes the images of reagent chips one by one through the window of the analyzer by a CCD camera, and then downloads the images into a computer. A conventional reagent chip analyzer has the capability to read only one specification of the reagent chips, because its slot can only allow one particular specification of reagent chips.

### SUMMARY OF THE INVENTION

The problem to be solved in the present invention is to overcome the shortcomings of a conventional analyzer that allows only one specification of reagent chip to insert into the slot. If there is more than one specification of reagent chips, several analyzers are needed. They occupy more space and are more expensive.

A primary objective of the present invention is to provide an analyzer, which may vary the width of a slot for the insertion of reagent chips. The analyzer adjusts the size of the slot according to various specifications of the reagent chips, in order to achieve a broader application.

Because only one USB transmission cable supplies power to the CCD or CMOS camera in accordance with the present invention, the power supply for the analyzer is limited. The advantage is that a computer can provide the enough power for the analyzer, or it is necessary to get a power source for driving the analyzer. Thus, the reagent chips will be inserted or ejected manually.

A technical feature of the present invention is to install a gear and two gear racks, which are symmetrically engaging the gear, on a base. The gear racks are connected with two guiding components respectively, which guide the reagent chip to enter the analyzer. Springs are installed between the gear racks and the base. When the reagent chips are inserted, the guiding components are pushed apart simultaneously. When reagent chips are ejected, the springs will force the guiding components close to each other.

Another technical feature of the present invention is to provide two positioning blocks and a positioning plate. The positioning plate with two positioning holes is mounted on the base. The positioning blocks are mounted on the guiding components and placed in the positioning holes. When the guiding components move back and forth, the moving ranges of the positioning blocks are limited by the positioning holes, thereby restricting the moving ranges of the guiding components.

The slot of the conventional analyzer only allows the insertion of one specification of reagent chips. In order to test and analyze various specifications of the reagent chips, it is necessary to prepare many analyzers corresponding to the specifications of the reagent chips. Thus it not only occupies a lot of space, but also costs a lot of money.

The present invention provides an analyzer being capable of adjusting the width of the slot according to various specifications of reagent chips, thereby improving application flexibility of various specifications of the reagent chips.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a perspective view of a reagent chip analyzer in accordance with the present invention;

FIG. 2 is a perspective exploded view showing the structure of the reagent chip analyzer in accordance with the present invention;

FIG. 3 is another perspective exploded view showing the structure of the reagent chip analyzer in accordance with the present invention;

FIG. 4 is a planar top view showing that two guiding components move away from each other as reagent chips are inserted;

FIG. 5 is a planar top view showing that two guiding components move close to each other as reagent chips are ejected; and

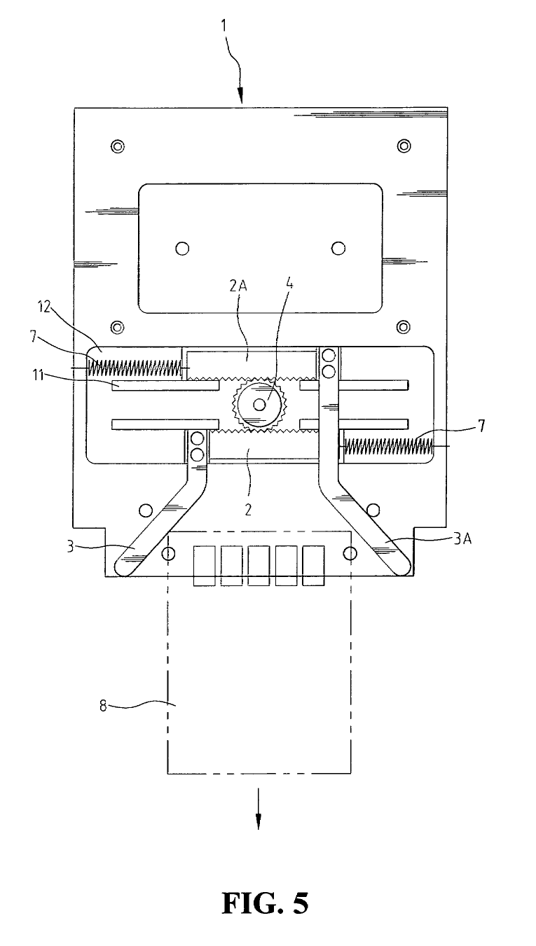

FIG. 6 is a cross-sectional side view showing the structure of the reagent chip analyzer in accordance with the present invention.

### DETAILED DESCRIPTION OF THE PREFERRED EMBODIMENT

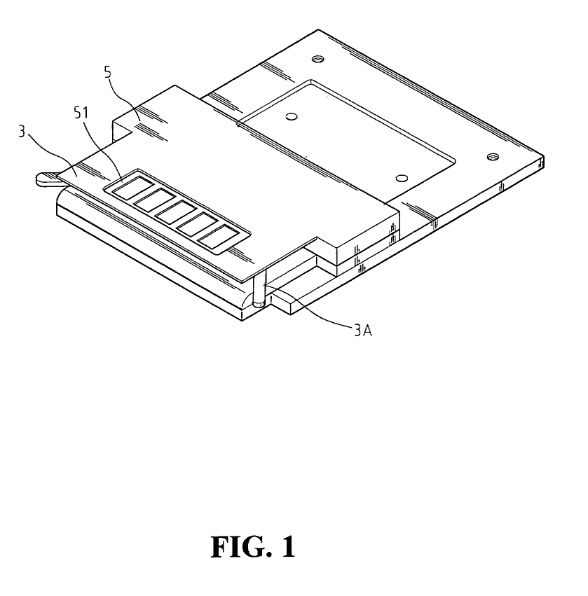

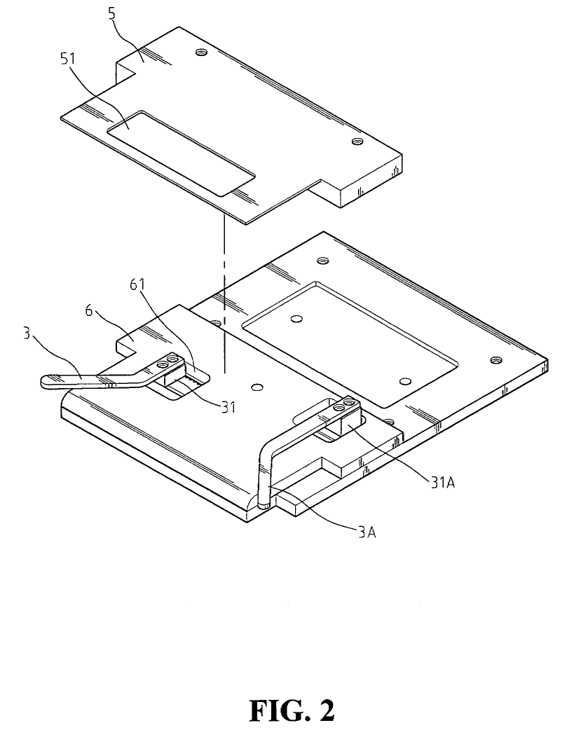

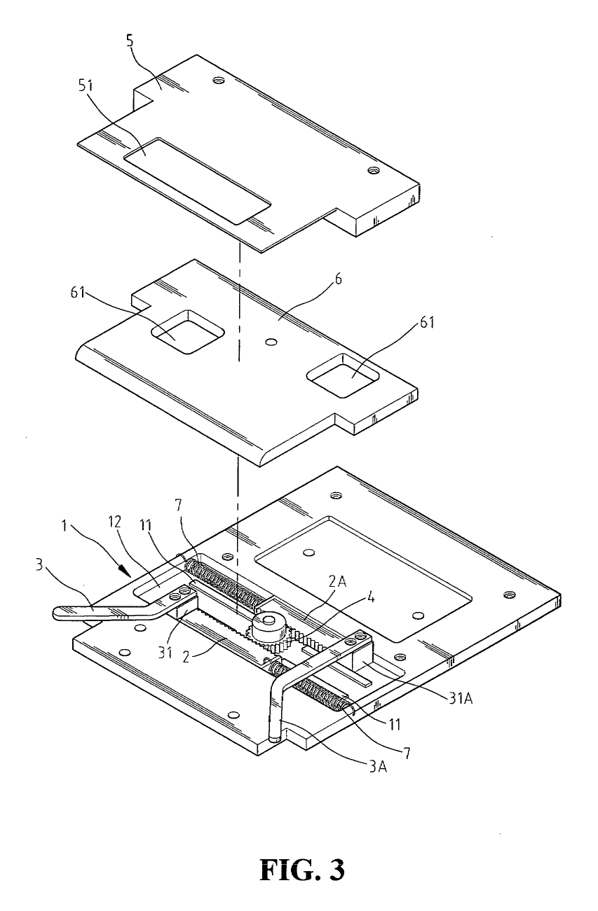

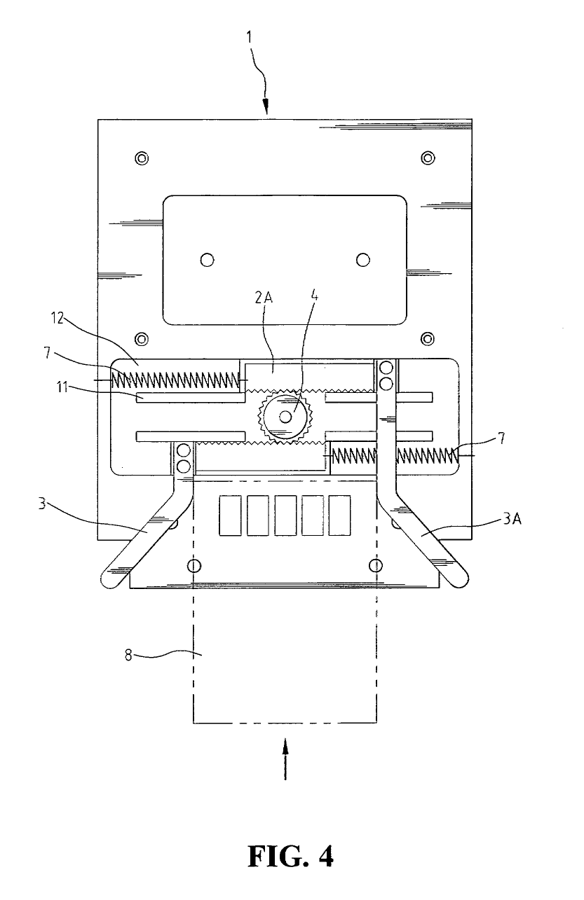

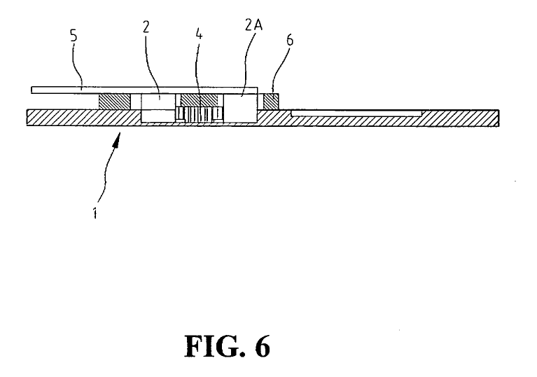

As shown in FIGS. 1, 2, 3, and 6, a cartridge-type reagent chips analyzer in accordance with the present invention comprises a base 1, a positioning plate 6, a cover plate 5, at least two bars 11, two grooves 12, a gear 4, two gear racks 2, 2A, springs 7, guiding components 3, 3A, and positioning blocks 31, 31A. The positioning plate 6 is mounted on the base 1, and the cover plate 5 is installed on the positioning plate 6. Referring to FIG. 4, a slot for the insertion of a reagent chip 8 is defined between the cover plate 5 and the front of the base 1. The parallel bars 11 are installed on the base 1. The grooves 12 are formed by the bars 11 and are parallel to the front edge of the base 1. The gear 4 is installed on the base 1 and between the grooves 12. The gear racks 2, 2A are installed in the grooves 12 respectively, and mesh with the gear 4 symmetrically. The springs 7 are installed between the gear racks 2, 2A and the lateral sides of the base 1, respectively. The guiding components 3, 3A are symmetrically installed on gear racks 2, 2A, respectively. The guiding components 3, 3A adopt a splayed structure according to the preferred embodiment of the present invention. The inner ends of the guiding components 3, 3A are parallel to each other, and the outer ends close to the entrance of the slot spread apart. The guiding components 3, 3A are the devices for guiding reagent chips to be inserted into the base 1. Therefore, the splayed ends of the guiding components 3, 3A help guide the reagent chips to be inserted into the base 1. The positioning blocks 31, 31A are installed between the guiding components 3, 3A and gear racks 2, and 2A.

The positioning plate 6 with two rectangular positioning holes 61 is mounted on the base 1, when the gear 4 and the gear racks 2, 2A have been installed and the guiding components 3, 3A have not been installed. The positioning blocks 31, 31A are attached to the guiding components 3, 3A, placed in the positioning holes 61, and installed on the gear rack 2, 2A, respectively. The cover plate 5 is mounted on the positioning plate 6. A space for the insertion of the reagent chips 8 is defined between the cover plate 5 and the base 1. The guiding components 3, 3A are used to guide the insertion of the reagent chips 8. The cover plate 5 provides a window 51. The analyzer provides an image receiver, such as a CCD camera (not shown in the Figures), to take the images of reagent chips 8 through the window after the reagent chips 8 are inserted.

As shown in FIG. 4, when the reagent chip 8 is inserted into the analyzer, it pushes two guiding components 3, 3A apart from each other. The guiding components 3, 3A guide the reagent chip to enter the analyzer. When the reagent chip 8 is ejected out of the analyzer, the springs 7 pull the gear racks 2, 2A and then bring the guiding components 3, 3A close to each other. Thus, the guiding components 3, 3A return to their original positions. The positioning blocked 31, 31A are attached to the guiding components 3, 3A, placed in the positioning holes 61, and installed on the gear rack 2, 2A, respectively. The moving ranges of the positioning blocks 31, 31A are restricted by the positioning holes 61. Therefore, the moving ranges of the guiding components 3, 3A are also defined by the positioning holes 61.

Although the present invention has been described with reference to the preferred embodiment thereof, it is apparent to those skilled in the art that a variety of modifications and changes may be made without departing from the scope of the present invention which is intended to be defined by the appended claims.

What is claimed is:

1. A cartridge-type reagent chip analyzer, comprising
a base covered with a cover plate, wherein a space for the insertion of a reagent chip is defined between said base and said cover plate;
a gear installed on said base;
two gear racks installed symmetrically and parallel to each other on said base, and meshing with said gear;
two guiding components symmetrically installed on said two gear racks, respectively, wherein a slot for the insertion of said reagent chip is defined between said guiding components; and
two springs connecting to said gear racks and lateral sides of said base respectively, thereby forcing said guiding components back to original positions when there is no external force applied on said guiding components.

2. The cartridge-type reagent chip analyzer as claimed in claim 1, further comprising:
two positioning blocks installed on said guiding components respectively; and
a positioning plate with two positioning holes provided between said cover plate and said base;
wherein the width of said positioning holes is larger than that of said positioning blocks, and said positioning blocks are placed in said positioning holes, respectively.
